(19) United States
(12) Patent Application Publication
(10) Pub. No.: US 2023/0391712 A1
(43) Pub. Date: Dec. 7, 2023

(54) PROCESS FOR THE AMMONOLYSIS OF BROMOALKANOIC ACIDS

(71) Applicant: Arkema France, Colombes (FR)

(72) Inventors: Jean-François DEVAUX, PIERRE-BENITE CEDEX (FR); Bernard PEES, MARSEILLE CEDEX 11 (FR)

(73) Assignee: Arkeme France, Colombes (FR)

(21) Appl. No.: 18/228,658

(22) PCT Filed: Jan. 31, 2022
(86) PCT No.: PCT/FR2022/050165

§ 371 Date: Jul. 31, 2023

(30) Foreign Application Priority Data
Feb. 2, 2021 (FR) FR2100997

(51) Int. Cl. C07C 227/08; C07C 227/40

(52) U.S. Cl. CPC C07C 227/08; C07C 227/40

(57) ABSTRACT

A process for the manufacture of ω-aminoalkanoic acid of formula NH₂—(CH₂)ₙ—COOH, in which n is an integer from 9 to 11, by reaction of the corresponding ω-bromoalkanoic acid with ammonia, including the following stages: i) reaction of the ω-bromoalkanoic acid with an aqueous excess ammonia solution, and ii) separation of the ω-aminoalkanoic acid formed from the reaction mixture. The aqueous ammonia solution exhibits a concentration of 35% to 70% by weight, and stage i) is carried out under a pressure greater than atmospheric pressure, from 0.11 to 2.0 MPa absolute.

### TECHNICAL FIELD

The present patent application relates to a process for the ammonolysis of ω-bromoalkanoic acids in order to manufacture the corresponding ω-aminoalkanoic acids, of use as monomers for polyamides.

### PRIOR ART

Processes for the ammonolysis of ω-bromoalkanoic acids of formula Br—(CH₂)ₙ—COOH, n=9 to 11, to give ω-aminoalkanoic acids NH₂—(CH₂)ₙ—CO₂H using ammonia are known.

The document FR 988 699 thus describes the reaction of 10-bromodecanoic acid with an aqueous 25% ammonia solution at 15° C. for 6 days in order to form 10-aminodecanoic acid with the yield of 77%. This process exhibits the disadvantage of a very long reaction time.

Increasing the temperature makes it possible to accelerate the reaction but promotes side reactions.

Thus, in the document FR 928 265, the reaction of 11-bromoundecanoic acid with an aqueous 25% ammonia solution carried out at 60° C. for 10 hours in a closed receptacle, and thus under autogenous pressure, is reported. The yield of this reaction is 53% and thus very low.

This is because, when the ammonolysis is carried out, the secondary amine of formula HO₂C—(CH₂)ₙ—NH—(CH₂)ₙ—CO₂H is in particular co-produced in a more or less large amount. This impurity is troublesome in the use of ω-aminoalkanoic acids for manufacturing polyamides as it brings about chain branchings. As this secondary amine reduces the yield and is difficult to separate from the desired primary amine, minimizing its formation is desired.

Patent Application CN 103804209 B describes, in Example 3, the ammonolysis of 11-bromoundecanoic acid with anhydrous ammonia under a pressure of 0.1 to 0.15 MPa in the presence of solvent and of a phase-transfer catalyst. This process makes it possible to reduce the reaction time to less than 24 hours but operates in a very dilute medium, and thus requires large industrial plants, and exhibits a still fairly modest yield, of 84.3%.

Patent Application EP 2 358 662 B1 proposes to reduce the reaction time for the ammonolysis of 11-bromoundecanoic acid and also the secondary reactions by carrying out the reaction with an increasing temperature profile, the reaction medium being subjected to a rise in temperature by regular stationary phases between an initial temperature of 15 to 25° C. and a final temperature of 26 to 40° C.

A search is still underway for technical solutions which make it possible to minimize the production of secondary amine while controlling the plant sizes.

### SUMMARY OF THE INVENTION

It is an aim of the invention to provide a solution to one or more of the abovementioned problems.

This is because the present invention is based on the unexpected observation that the increase in the titre of the aqueous ammonia solution above 35% by weight, in combination with an increase in the pressure above atmospheric pressure, makes it possible to accelerate the reaction while controlling the amount of secondary amine produced and thus to increase the yield.

Consequently, according to a first aspect, a subject-matter of the invention is a process for the manufacture of ω-aminoalkanoic acid of formula NH₂—(CH₂)ₙ—COOH, in which n is an integer from 9 to 11, by reaction of the corresponding bromoalkanoic acid with ammonia, comprising the following stages:
i) reaction of the ω-bromoalkanoic acid with an aqueous excess ammonia solution,
ii) separation of the ω-aminoalkanoic acid formed from the reaction mixture, characterized in that the aqueous ammonia solution exhibits a concentration of 35% to 70% by weight and that stage i) is carried out under a pressure greater than atmospheric pressure, from 0.11 to 2.0 MPa absolute.

Preferably, stage i) is carried out at a temperature of between 0 and 60° C. It can be carried out at a constant temperature or at an increasing temperature. When it is carried out at an increasing temperature, stage i) can advantageously be carried out with an initial temperature ranging from 0 to 25° C. and a final temperature ranging from 26 to 60° C.

Preferably, the aqueous ammonia solution exhibits a concentration of 36% to 60%, preferably of 38% to 45%, by weight.

Advantageously, the ratio by weight of the ω-bromoalkanoic acid at the start of stage i) to the aqueous ammonia solution is from 1:3 to 1:20.

Stage i) of the process according to the invention can be carried out in particular under a pressure of 0.125 to 0.5 MPa absolute, or also of 0.13 to 0.3 MPa absolute.

The process of the invention can be carried out continuously or batchwise.

Advantageously, the process according to the invention additionally comprises a stage iii) of washing and draining the ω-aminoalkanoic acid obtained on conclusion of stage ii).

It can also additionally comprise a stage iv) of purification of the crude ω-aminoalkanoic acid obtained on conclusion of stage ii) or iii), preferably by recrystallization from boiling water.

It can furthermore also comprise, in addition, a stage v) of recovery of the residual w-aminoalkanoic acid in the various filtrates and aqueous washing liquors obtained in stages ii), iii) and/or iv), in particular by degassing the ammonia, liquid-liquid extraction, crystallization, filtration and washing.

Finally, the process of the invention can additionally comprise a stage vi) of drying the w-aminoalkanoic acid obtained.

### DESCRIPTION OF THE EMBODIMENTS

### Definition of the Terms

Throughout this disclosure, the percentages are, unless otherwise mentioned, understood as being percentages by weight with respect to the composition under consideration.

Furthermore, the pressure values given in the present disclosure are, unless otherwise mentioned, understood as being absolute pressure values, that is to say with reference to the absolute vacuum.

The term “aqueous ammonia solution” is understood to mean a solution of NH₃ in water. Its ammonia content is expressed as weight of NH₃ with respect to the weight of solution (NH₃ and water).

According to the present invention, the process for the manufacture of ω-aminoalkanoic acid of formula NH₂—(CH₂)ₙ—COOH, in which n is an integer from 9 to 11, by reaction of the corresponding ω-bromoalkanoic acid with ammonia, thus comprises the following stages:
i) reaction of the ω-bromoalkanoic acid with an aqueous excess ammonia solution,
ii) separation of the ω-aminoalkanoic acid formed from the reaction mixture,

characterized in that the aqueous ammonia solution exhibits a concentration of 35% to 70% by weight and that stage i) is carried out under a pressure greater than atmospheric pressure, from 0.11 to 2.0 MPa absolute. The ω-bromoalkanoic acid can be 10-bromodecanoic acid, 11-bromoundecanoic acid or 12-bromododecanoic acid.

These ω-bromoalkanoic acids can be obtained in particular by homobromination reaction of the corresponding unsaturated acid, namely 9-decenoic, 10-undecenoic or 11-dodecenoic acid. These compounds are commercially available. 9-Decenoic acid can furthermore be obtained from oleic-based vegetable oil according to the process described in WO 2018/080869. 10-Undecenoic acid can be obtained from castor oil according to FR 928 265.

The name of ω-bromoalkanoic acid is used to denote said compound, in the more or less purified form, thus including its possible impurities. The commercial ω-bromoalkanoic acid generally has a purity of greater than 98%. Nevertheless, according to one embodiment, the purity of the ω-bromoalkanoic acid employed can also be between 90% and 98% and preferably between 93% and 96%. The ω-bromoalkanoic acid can contain, as impurity, in particular the bromoalkanoic acid of formula CH₃—CH₂Br—(CH₂)ₙ₋₂—CO₂H.

The process of the invention comprises first of all a stage i) in which the ω-bromoalkanoic acid is mixed with the aqueous excess ammonia solution and then it is reacted.

The ω-bromoalkanoic acid can be added in the solid form or in the liquid form, in particular in the molten state. The solid form can be a powder, granules or flakes. Advantageously, the ω-bromoalkanoic acid is used in the molten state, preferably at a temperature of from 5 to 60° C., preferably 10 to 40° C. and in particular 15 to 30° C. above its melting point.

According to the invention, the aqueous ammonia solution used contains from 35% to 70% by weight of ammonia. Preferably, it contains from 36% to 60% and more preferably from 38% to 45% by weight of ammonia. According to one embodiment, the ammonia content of the aqueous ammonia solution can be from 35% to 40%, or from 40% to 45%, or from 45% to 50%, or from 50% to 55%, or from 55% to 60%, or from 60% to 65% or also from 65% to 70% by weight.

Advantageously, a portion of this aqueous ammonia solution originates from the recovery of the excess ammonia and of the ammonium present in the reaction medium at the outlet of the present process.

Advantageously, the aqueous ammonia solution introduced into the reactor exhibits a temperature which is not greater than 25° C. Preferably, it is cooled to a temperature of between −20° C. and 20° C. and preferably a temperature of between −10 and 10° C.

The ratio by weight of the ω-bromoalkanoic acid to the aqueous ammonia solution introduced in stage i) is such that the ammonia is present in stoichiometric excess. This excess does not promote only the reaction but also makes it possible to disperse or dilute the ω-bromoalkanoic acid and the product formed. Generally, it is between 1:20 and 1:3, preferably between 1:10 and 1:4, and preferably between 1:8 and 1:4. The term “ratio by weight” here denotes both the ratio of the weights of reactants used in the case of a batch process and the ratio of the flow rates by weight of the reactants in the case of a continuous process.

The process of the invention furthermore comprises a stage ii) in which the w-bromoalkanoic acid formed is separated from the reaction mixture.

The reaction mixture is generally non-homogeneous. It can in particular comprise a gas phase, one or more liquid phases and/or one or more solid phases.

The process of the invention can be carried out continuously or batchwise.

When the process is carried out continuously, the mixing of the reactants can be carried out in a tank provided with a stirrer. In an alternative form, it can also be carried out in an external mixing device, for example a static mixer, a Venturi device or a recirculation loop into which the ω-bromoalkanoic acid is injected.

Stage i) of the process can be carried out in the same device. According to one embodiment, stage i) can be carried out in one or more stirred tanks. According to a preferred embodiment, stage i) is carried out in a battery of 2 to 25 reactors connected in series, in which the reaction mixture is sent from one reactor to the other using a pump or by gravity flow. Each reactor can consist of a tank provided with stirring. This stirring can be produced by stirring modules inside the reactor or by external recirculation.

Preferably, stage i) is carried out at a temperature of 0 to 60° C. According to one embodiment, the temperature can be from 0 to 5° C., or from 5 to 10° C., or from 10 to 15° C., or from 15 to 20° C., or from 20 to 25° C., or from 25 to 30° C., or from 30 to 35° C., or from 35 to 40° C., or from 40 to 45° C., or from 45 to 50° C., or from 50 to 55° C., or also from 55 to 60° C.

The temperature of the reactor(s) can advantageously be controlled by the circulation of heat-transfer fluid in a jacket of the reactor(s) or heat exchangers in the reactor(s) or outside the reactor(s) or by injection of a hot fluid, in particular water or steam, into one or more reactors.

The temperature can also be controlled by the evaporation of a portion of the ammonia in order to cool the reaction medium, as explained in more detail later.

When the process is carried out in a battery of reactors, as explained above, the temperature in the first reactor is preferably from 0 to 25° C., and that in the final reactor from 26 to 60° C., and the temperature increases from reactor to reactor. In such a case, it is advantageous to provide individual control of the temperature in each reactor. Advantageously, the first reactor(s) can be cooled and the final reactor(s) can be heated.

The total residence time for stage i) (calculated as the ratio of the sum of the volumes of liquid phase in the reactors to the sum of the flow rates of reactants) is generally from 20 to 100 hours and preferably from 40 to 80 hours.

According to a second alternative embodiment, stage i) of the process is carried out batchwise in a reactor which can be a tank provided with stirring means. According to one embodiment, the reactants are mixed outside the reactor and then introduced as a mixture into the reactor. According to another embodiment, the ammonia solution is introduced first into the reactor and then the ω-bromoalkanoic acid is added.

The mixing of the ω-bromoalkanoic acid in the aqueous ammonia solution can be carried out in a tank provided with a stirrer. In an alternative form, the mixing can be carried out by means of a mixing device external to the reactor, such as, for example, a static mixer, or of a Venturi device which makes it possible to mix the two streams of reactants in line before they are injected into the reactor, or also of a recirculation loop external to the reactor into which the ω-bromoalkanoic acid is injected.

After mixing the reactants, stage i) consists in leaving the reaction medium to stir for a sufficient period of time, generally from 20 to 100 hours and preferably 40 to 80 hours.

According to one embodiment, the reaction of stage i) is carried out isothermally, that is to say with a temperature regulated at one and the same value throughout the duration of the reaction.

In this case, stage i) is carried out at a temperature of 0 to 60° C. According to one embodiment, the temperature can be from 0 to 5° C., or from 5 to 10° C., or from 10 to 15° C., or from 15 to 20° C., or from 20 to 25° C., or from 25 to 30° C., or from 30 to 35° C., or from 35 to 40° C., or from 40 to 45° C., or from 45 to 50° C., or from 50 to 55° C., or also from 55 to 60° C.

According to another embodiment, the reaction is carried out in increasing temperature mode. Among the different thermal profiles which can be envisaged, it has proved advantageous to provide stationary phases at increasing temperatures, in particular ranging from an initial temperature of 0 to 25° C. up to a final temperature of 26 to 60° C.

According to an alternative embodiment, stage i) is carried out in a battery of reactors in parallel R1, R2, . . . Rn, 2≤n≤25, which are independently maintained at a variable temperature. Each reactor has available a program for rise in temperature over a given period of time, which period of time depends on the temperatures chosen. In each reactor, the initial temperature of the program is preferentially between 0 and 25° C. and the final temperature from 26 to 60° C. In order to keep feeding and withdrawing constant, regular and continuous at the inlet and at the outlet of the battery of reactors, each reactor operates according to charging/reaction/emptying cycles with an offset in time equivalent to the reaction time divided by the number of reactors (n).

In all the alternative forms, the pressure in the reactor(s), measured at the top point, generally in a gas head space, is kept above atmospheric pressure during stage i). This is because this makes it possible to maintain a high concentration of ammonia in the liquid reaction phase, which promotes a high selectivity for primary amine. It is thus possible to limit the production of impurities, in particular of secondary amines, indeed even tertiary amines. It will nevertheless be preferred to limit the pressure in order to limit the costs of the reactors.

Consequently, the pressure in the reactor(s) is between 0.11 and 2.0 MPa absolute and preferably from 0.125 to 0.5 MPa absolute and preferably from 0.13 to 0.3 MPa absolute. According to one embodiment, the pressure can be from 0.11 to 0.15 MPa absolute, or from 0.15 to 0.2 MPa absolute, or from 0.2 to 0.25 MPa absolute, or from 0.25 to 0.3 MPa absolute, or from 0.3 to 0.35 MPa absolute, or from 0.35 to 0.4 MPa absolute, or from 0.4 to 0.45 MPa absolute, or from 0.45 to 0.5 MPa absolute, or from 0.5 to 0.55 MPa absolute, or from 0.55 to 0.6 MPa absolute, or from 0.6 to 0.65 MPa absolute, or from 0.65 to 0.7 MPa absolute, or from 0.7 to 0.75 MPa absolute, or from 0.75 to 0.8 MPa absolute, or from 0.8 to 0.85 MPa absolute, or from 0.85 to 0.9 MPa absolute, or from 0.9 to 0.95 MPa absolute, or from 0.95 to 1.0 MPa absolute, or from 1.0 to 1.05 MPa absolute, or from 1.05 to 1.1 MPa absolute, or from 1.10 to 1.15 MPa absolute, or from 1.15 to 1.2 MPa absolute, or from 1.2 to 1.25 MPa absolute, or from 1.25 to 1.3 MPa absolute, or from 1.3 to 1.35 MPa absolute, or from 1.35 to 1.4 MPa absolute, or from 1.4 to 1.45 MPa absolute, or from 1.45 to 1.5 MPa absolute, or from 1.5 to 1.55 MPa absolute, or from 1.55 to 1.6 MPa absolute, or from 1.6 to 1.65 MPa absolute, or from 1.65 to 1.7 MPa absolute, or from 1.7 to 1.75 MPa absolute, or from 1.75 to 1.8 MPa absolute, or from 1.8 to 1.85 MPa absolute, or from 1.85 to 1.9 MPa absolute, or from 1.9 to 1.95 MPa absolute, or from 1.95 to 2.0 MPa absolute.

When stage i) is carried out in several reactors, the vents of the reactors can be connected so that they operate at virtually identical pressures. According to another embodiment, it can be advantageous to places the reactors under a different pressure. Thus, when the temperature is conducted in a way increasing from reactor to reactor, as explained above, the pressure in the reactors can also be increased from reactor to reactor. According to one embodiment, the pressure prevailing in the reactor(s) is the autogenous pressure, imposed mainly by the vapour pressure of the ammonia related to the composition of the reaction mixture and its temperature.

According to one embodiment, the evaporation of a portion of the ammonia is made possible in order to maintain the temperature of the reaction medium. Thus, when operating continuously, it is possible to cool the first reactor(s) by this means. When operating batchwise, it is possible to cool the reactor by this means at the reaction start. It is possible either to regulate a flow rate of evaporation of ammonia, and in this case the pressure is dealt with, or else to impose a pressure slightly lower than the liquid-vapour equilibrium and to deal with the evaporation of ammonia. Advantageously, between 0.1% and 50% and preferably from 10% to 30% of the ammonia injected is evaporated during stage i). A person skilled in the art knows how to adjust the amount of ammonia to be evaporated from the reaction medium in order to maintain the temperature profile during the reaction. This mode of operation makes it possible to limit or to avoid expensive heat-exchange devices while providing the temperature profile which makes possible optimum yields.

The process of the invention thus makes it possible to greatly limit the amount of secondary amine formed and to consequently increase the yield of the reaction.

According to the invention, the process additionally comprises a stage ii) of separation of the ω-aminoalkanoic acid from the reaction mixture on conclusion of stage i).

The ω-aminoalkanoic acid formed on conclusion of stage i) can be separated from the reaction mixture in a way conventional per se, for example by the following stages.

The reaction mixture resulting from stage i) can be diluted in water and heated to boiling. The ammonia which is given off is scrubbed out in water to form an aqueous ammonia solution, which can be recycled to stage i) of the present process. The reaction mixture depleted in ammonia can subsequently be separated from the possible oil layer formed by separation by settling under hot conditions before being cooled in order to separate the w-aminoalkanoic acid by crystallization and solid-liquid separation.

Furthermore, the filtrates rich in ammonium bromide recovered from the solid-liquid separation can be treated, for example with sodium hydroxide, for the purpose of reforming the ammonia, which is evaporated and then scrubbed out in water, and of obtaining an aqueous ammonia solution.

The ω-aminoalkanoic acid can also be separated from the reaction mixture obtained on conclusion of stage i) by a stripping stage in order to remove the ammonia, which is scrubbed out in water, followed by solid-liquid separation, such as filtration on a filter or draining. The mother liquors recovered can undergo a liquid-liquid extraction, a crystallization and/or a filtration in order to form an aqueous solution rich in ammonium bromide and depleted in ω-aminoalkanoic acid. This aqueous solution rich in ammonium bromide can be treated with sodium hydroxide to reform ammonia, which is evaporated and then scrubbed out in water to form an aqueous ammonia solution.

The process of the invention can also comprise an additional stage iii), in which the w-aminoalkanoic acid obtained in stage ii) is subjected to washing and draining. The washing can in particular be carried out with water.

The process of the invention can also additionally comprise a stage iv) of purification of the crude ω-aminoalkanoic acid obtained on conclusion of stage ii) or iii).

This purification stage can in particular comprise one or more of the following operations: recrystallization in an appropriate solvent, such as water or a basic or acidic aqueous solution, such as a mixture of water and carboxylic acid, filtration, washing and draining.

Preferably, the crude ω-aminoalkanoic acid obtained is purified by recrystallization from very hot water.

Consequently, the process of the invention can additionally comprise a stage v) of recovery of the residual ω-aminoalkanoic acid in the various filtrates and aqueous washing liquors obtained in stages ii), iii) and/or iv), in particular by degassing the ammonia, liquid-liquid extraction, crystallization, filtration and washing.

Finally, the process of the invention can additionally comprise a stage vi) of drying the w-aminoalkanoic acid obtained.

Advantageously, the process according to the invention makes it possible to obtain an w-aminoalkanoic acid in which the content of ω-aminodialkanoic acid, with respect to the content of ω-aminoalkanoic acid, is, on conclusion of stage i) and thus before purification, less than 1.8% by weight, preferably less than 1.5% by weight.

Furthermore, the process according to the invention advantageously makes possible complete conversion of the ω-bromoalkanoic acid over the duration of the ammonolysis stage i), in a reaction time of less than 75 h.

The invention will be explained in more detail in the examples which follow.

### EXAMPLES

### Example 1

660 g of an aqueous 40% by weight ammonia solution cooled to 0° C. are placed in a one-litre autoclave equipped with a mechanical stirrer having two propellers having five blades and with a turbine of Rushton type rotating at 400 rpm and with a coil making possible the circulation of a heat-transfer fluid and with a discharge valve set at 0.15 MPa absolute and then the reactor is closed. 110 g of molten 11-bromoundecanoic acid (purity 98%) at 90° C. are rapidly added dropwise and then the temperature of the reaction medium is adjusted to 22° C. by virtue of the heat-transfer fluid circulating in the coil. The pressure very rapidly adjusts to 0.15 MPa absolute and remains at this value throughout the duration of the reaction. After 12 h 30, the temperature of the reaction medium is increased in order to successively carry out stationary phases of 12 h 30 respectively at 24, 26, 28, 30 and 32° C. The vent of the reactor is subsequently opened in order to bring the reactor to atmospheric pressure.

The reaction medium is then analysed. By quantitatively determining the bromide ions with silver nitrate using a silver electrode, it is found that all of the 11-bromoundecanoic acid has reacted. By an HPLC quantitative determination with external calibration, 1.3% by weight of 11-aminodiundecanoic acid are measured, with respect to the 11-aminoundecanoic acid.

### Example 2 (Comparative Example)

Example 1 is repeated but while operating at atmospheric pressure throughout the duration of the experiment and while controlling the rate of addition of molten 11-bromoundecanoic acid in order for the medium not to foam uncontrollably.

The analysis of the reaction medium shows that all of the 11-bromoundecanoic acid has reacted. 1.9% by weight of 11-aminodiundecanoic acid are measured, with respect to the 11-aminoundecanoic acid.

### Example 3 (Comparative Example)

Example 1 is repeated but while operating at atmospheric pressure throughout the duration of the experiment and while using 660 g of an aqueous 32% by weight ammonia solution.

The analysis of the reaction medium shows that all of the 11-bromoundecanoic acid has reacted. 2.0% by weight of 11-aminodiundecanoic acid are measured, with respect to the 11-aminoundecanoic acid.

### LIST OF THE DOCUMENTS CITED

FR 988 699
WO 2018/080869 A1
FR 928 265
CN 103804209 B
EP 2 358 662 B1

1. A process for the manufacture of ω-aminoalkanoic acid of formula NH₂—(CH₂)ₙ—COOH, in which n is an integer from 9 to 11, by reaction of the corresponding ω-bromoalkanoic acid with ammonia, comprising the following stages:
i) reaction of the ω-bromoalkanoic acid with an aqueous excess ammonia solution, and
ii) separation of the ω-aminoalkanoic acid formed from the reaction mixture, wherein the aqueous ammonia solution exhibits a concentration of 35% to 70% by weight, stage i) is carried out under a pressure greater than atmospheric pressure, from 0.11 to 2.0 MPa absolute.

2. The process according to claim 1, in which stage i) is carried out at a temperature of between 0 and 60° C.

3. The process according to claim 2, in which stage i) is carried out at a constant temperature.

4. The process according to claim 2, in which stage i) is carried out at an increasing temperature.

5. The process according to claim 4, in which stage i) is carried out at an increasing temperature, the initial temperature ranging from 0 to 25° C. and the final temperature ranging from 26 to 60° C.

6. The process according to claim 1, in which the aqueous ammonia solution exhibits a concentration of 36% to 60% by weight.

7. Three process according to claim 1, in which the ratio by weight of the ω-bromoalkanoic acid at the start of stage i) to the aqueous ammonia solution is from 1:3 to 1:20.

8. The process according to claim 1, in which the process is carried out under a pressure of 0.125 to 0.5 MPa absolute.

9. The process according to claim 8, in which the process is carried out under a pressure of 0.13 to 0.3 MPa absolute.

10. The process according to claim 1, carried out continuously.

11. The process according to claim 1, carried out batchwise.

12. The process according to claim 1, additionally comprising a stage iii) of washing and draining the ω-aminoalkanoic acid obtained on conclusion of stage ii).

13. The process according to claim 12, additionally comprising a stage iv) of purification of the crude ω-aminoalkanoic acid obtained on conclusion of stage ii) or iii).

14. The process according to claim 12, additionally comprising a stage v) of recovery of the residual ω-aminoalkanoic acid in the various filtrates and aqueous washing liquors obtained in stages ii), iii) and/or iv), in particular by degassing the ammonia, liquid-liquid extraction, crystallization, filtration and washing.

15. The process according to claim 1, additionally comprising a stage vi) of drying the ω-aminoalkanoic acid obtained.
